(19) United States
(12) Patent Application Publication
(10) Pub. No.: US 2015/0240292 A1
(43) Pub. Date: Aug. 27, 2015

(54) NUCLEIC ACID AMPLIFICATION METHOD, NUCLEIC ACID EXTRACTION DEVICE, NUCLEIC ACID AMPLIFICATION REACTION CARTRIDGE, AND NUCLEIC ACID AMPLIFICATION REACTION KIT

(71) Applicant: Seiko Epson Corporation, Tokyo (JP)

(72) Inventor: Masato HANAMURA, Shiojiri (JP)

(21) Appl. No.: 14/628,706

(22) Filed: Feb. 23, 2015

(30) Foreign Application Priority Data
Feb. 27, 2014 (JP) 2014-036415

(51) Int. Cl. C12Q 1/68

(52) U.S. Cl. CPC C12Q 1/6806

(57) ABSTRACT

A nucleic acid amplification method includes: adsorbing a nucleic acid on fine particles by mixing an adsorbing liquid and the fine particles with a sample containing the nucleic acid; washing the fine particles having the nucleic acid adsorbed thereon with a first washing liquid; eluting the nucleic acid adsorbed on the fine particles in an eluate by applying ultrasound to the fine particles having the nucleic acid adsorbed thereon; and performing a nucleic acid amplification reaction of the nucleic acid in the eluate. Further provided are a nucleic acid extraction device, a nucleic acid amplification reaction cartridge, and a nucleic acid amplification reaction kit, capable of realizing this method.

### BACKGROUND

1. Technical Field

The present invention relates to a nucleic acid amplification method, a nucleic acid extraction device, a nucleic acid amplification reaction cartridge, and a nucleic acid amplification reaction kit.

2. Related Art

In recent years, as a result of development of technologies utilizing genes, medical treatments utilizing genes such as genetic diagnosis or genetic therapy are drawing attention. In addition, many methods utilizing genes in determination of breed varieties or breed improvement have also been developed in agricultural and livestock industries. As technologies for utilizing genes, PCR (Polymerase Chain Reaction) and the like are widely used. Nowadays, PCR has become an indispensable technology in elucidation of information on biological materials. PCR is a method of amplifying a target nucleic acid by subjecting a solution (a reaction mixture) containing a nucleic acid to be amplified (a target nucleic acid) and a reagent to a thermal cycle. In the thermal cycle of PCR, a method of performing a thermal cycle at two or three temperature stages is generally used.

On the other hand, the diagnosis of an infectious disease as represented by influenza in medical practice is currently carried out mainly by using a simple test kit such as an immunochromatograph. However, such a simple test sometimes provides insufficient accuracy, and the application of PCR, which can be expected to provide higher test accuracy, to the diagnosis of an infectious disease has been eagerly awaited. Further, in general outpatient practice or the like in a medical institution, because the consultation time is limited, the time that can be spent for testing is limited to a short period. Due to this, the current situation is that, for example, the diagnosis of influenza is carried out by a simple test using an immunochromatograph or the like in a shorter time at the sacrifice of test accuracy.

In light of such circumstances, in order to realize a test using PCR, which can be expected to provide higher test accuracy, in medical practice, it was necessary to reduce a time required for the reaction. As an apparatus for performing the PCR reaction in a short time, for example, JP-A-2009-136250 (PTL 1) discloses a biological sample reaction apparatus which performs a thermal cycle by rotating a biological sample reaction chip filled with a reaction mixture and a liquid which is immiscible with the reaction mixture and has a specific gravity smaller than that of the reaction mixture about the rotation axis in the horizontal direction so as to move the reaction mixture. Further, there have been disclosed as the method for PCR, the following methods, etc.: a method using magnetic beads (JP-A-2009-207459 (PTL 2)) and a method of performing a PCR thermal cycle by moving a liquid droplet in a temperature variable region on a substrate using magnetic beads as a liquid droplet moving unit (JP-A-2008-012490 (PTL 3)).

In a method of performing a nucleic acid amplification reaction at a high speed by adsorbing a nucleic acid on fine particles disclosed in PTL 2 and PTL 3, a step of eluting the nucleic acid from the fine particles follows, and there has been a demand that this step is performed efficiently in a short time.

### SUMMARY

An advantage of some aspects of the invention is to provide a nucleic acid amplification method having higher elution efficiency, and also provide a nucleic acid extraction device, a nucleic acid amplification reaction cartridge, and a nucleic acid amplification reaction kit, capable of realizing this method.

A nucleic acid amplification method according to an aspect of the invention includes: adsorbing a nucleic acid on fine particles by mixing an adsorbing liquid and the fine particles with a sample containing the nucleic acid; washing the fine particles having the nucleic acid adsorbed thereon with a first washing liquid; eluting the nucleic acid adsorbed on the fine particles in an eluate by applying ultrasound to the fine particles having the nucleic acid adsorbed thereon; and performing a nucleic acid amplification reaction of the nucleic acid in the eluate.

A nucleic acid amplification method according to another aspect of the invention includes: adsorbing a nucleic acid on fine particles by mixing an adsorbing liquid and the fine particles with a sample containing the nucleic acid; washing the fine particles having the nucleic acid adsorbed thereon with a second washing liquid; washing the fine particles having the nucleic acid adsorbed thereon after being washed with the second washing liquid with a first washing liquid; eluting the nucleic acid adsorbed on the fine particles in an eluate by applying ultrasound to the fine particles having the nucleic acid adsorbed thereon; and amplifying the nucleic acid.

In either of the nucleic acid amplification methods described above, the nucleic acid may be a ribonucleic acid (RNA), the method may further include reverse-transcribing the ribonucleic acid eluted in the eluate, thereby synthesizing a cDNA, and the nucleic acid to be amplified in the nucleic acid amplification may be the synthesized cDNA.

A nucleic acid extraction device according to still another aspect of the invention includes: a tube having a longitudinal direction and internally provided, in the following order, with a first plug composed of an oil, a second plug composed of a first washing liquid, which is phase-separated from an oil, and with which fine particles having a nucleic acid bound thereto are washed, a third plug composed of an oil, a fourth plug composed of an eluent, which is phase-separated from an oil, and with which the nucleic acid is eluted from the fine particles having the nucleic acid bound thereto, and a fifth plug composed of an oil; an ultrasound generating section which applies ultrasound to the fine particles having the nucleic acid bound thereto; and a vessel which can be connected to and communicate with the tube on the side where the first plug is disposed, and contains an adsorbing liquid for adsorbing the nucleic acid on the fine particles.

A nucleic acid extraction device according to yet another aspect of the invention includes: a tube having a longitudinal direction and internally provided, in the following order, with a first plug composed of an oil, a second plug composed of a first washing liquid, which is phase-separated from an oil, and with which fine particles having a nucleic acid bound thereto are washed, a third plug composed of an oil, a fourth plug composed of an eluent, which is phase-separated from an oil, and with which the nucleic acid is eluted from the fine particles having the nucleic acid bound thereto, and a fifth plug composed of an oil; an ultrasound generating section which performs an ultrasound treatment of the fine particles having the nucleic acid bound thereto; and a liquid reservoir section which communicates with the tube on the side where the first plug is disposed, and contains an adsorbing liquid for adsorbing the nucleic acid on the fine particles, wherein the adsorbing liquid contains an alcohol, and the first washing liquid is acidic.

A nucleic acid extraction device according to still yet another aspect of the invention includes: a tube having a longitudinal direction and internally provided, in the following order, with a first plug composed of an oil, a sixth plug composed of a second washing liquid, which is phase-separated from an oil, and with which fine particles having a nucleic acid bound thereto are washed, a seventh plug composed of an oil, a second plug composed of a first washing liquid, which is phase-separated from an oil, and with which the fine particles having the nucleic acid bound thereto and washed with the second washing liquid are washed, a third plug composed of an oil, a fourth plug composed of an eluent, which is phase-separated from an oil, and with which the nucleic acid is eluted from the fine particles having the nucleic acid bound thereto, and a fifth plug composed of an oil; and an ultrasound generating section which applies ultrasound to the fine particles having the nucleic acid bound thereto.

In any of the nucleic acid extraction devices described above, the device may further include a vessel which can be connected to the tube on the side where the first plug is disposed, and contains an adsorbing liquid for adsorbing the nucleic acid on the fine particles. Alternatively, the device may include a liquid reservoir section which communicates with the tube on the side where the first plug is disposed, and contains an adsorbing liquid for adsorbing the nucleic acid on the fine particles.

A nucleic acid amplification reaction cartridge according to further another aspect of the invention includes: the nucleic acid extraction device according to the aspect of the invention; and a nucleic acid amplification reaction vessel, which communicates with the tube on the side where the fifth plug is disposed, and contains an oil. The cartridge may further include a tank, which communicates with the tube on the side where the first plug is disposed, and from which the fine particles are introduced into the tube.

A nucleic acid amplification reaction kit according to still further another aspect of the invention includes: the nucleic acid extraction device according to the aspect of the invention; and a tank from which the fine particles are introduced into the tube.

### BRIEF DESCRIPTION OF THE DRAWINGS

The invention will be described with reference to the accompanying drawings, wherein like numbers reference like elements.

FIG. 1 is a view schematically showing a principal part of a nucleic acid extraction device according to an embodiment.

FIG. 2 is a view schematically showing a principal part of a nucleic acid extraction device according to an embodiment.

FIG. 3 is a view schematically showing a principal part of a nucleic acid extraction device according to an embodiment.

FIG. 4A is a view schematically showing a nucleic acid extraction device according to an embodiment.

FIG. 4B is a view schematically showing a nucleic acid extraction device according to an embodiment.

FIG. 5 is a view schematically showing a principal part of a nucleic acid extraction device according to an embodiment.

FIGS. 6A and 6B are views schematically showing a nucleic acid amplification reaction vessel of a nucleic acid amplification reaction cartridge according to an embodiment.

FIG. 7A is a view schematically showing one example of a nucleic acid extraction kit according to an embodiment.

FIG. 7B is a view schematically showing one example of a nucleic acid extraction kit according to an embodiment.

FIG. 8 is a view schematically showing one example of a nucleic acid amplification reaction cartridge according to an embodiment.

FIG. 9 is a schematic view for illustrating a modification of a nucleic acid extraction method according to an embodiment.

FIG. 10 is a graph showing the results of PCR performed using nucleic acids extracted in Examples.

### DESCRIPTION OF EXEMPLARY EMBODIMENTS

Hereinafter, several embodiments of the invention will be described. The embodiments described below are mere examples of the invention. The invention is by no means limited to the embodiments described below and also encompasses various modifications carried out within the scope in which the gist of the invention is not changed. Incidentally, not all structures described below are necessarily essential components of the invention.

### 1. NUCLEIC ACID EXTRACTION DEVICE

A nucleic acid extraction device 1000 according to this embodiment includes a tube section 100, a first plug 10, a second plug 20, a third plug 30, a fourth plug 40, a fifth plug 50, and an ultrasound generating device as an ultrasound generating section for performing an ultrasound treatment of fine particles having a nucleic acid bound thereto.

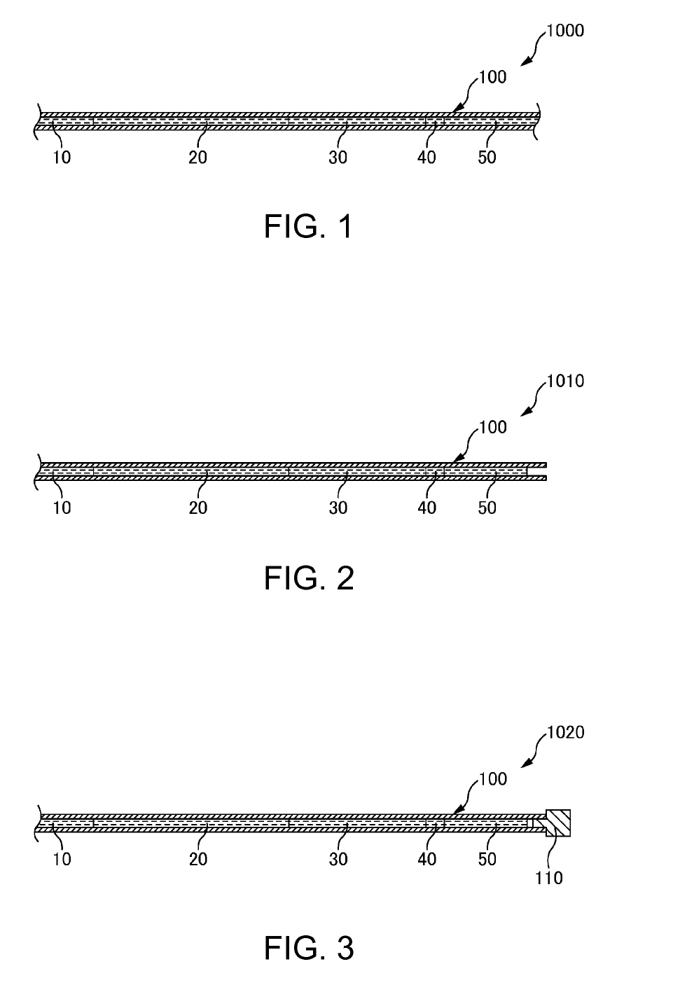

FIG. 1 is a view schematically showing a principal part of a nucleic acid extraction device 1000 according to this embodiment.

### 1.1. Tube Section

The tube section 100 constitutes the principal part of the nucleic acid extraction device 1000. The nucleic acid extraction device 1000 may include various structures other than the tube section 100. The nucleic acid extraction device 1000 may include, for example, a pipe, a vessel, a stopper, a joint, a pump, a control device, or the like to be connected to the tube section 100 although not shown in the drawing.

The tube section 100 is a cylindrical part having a hollow interior portion and capable of allowing a liquid to flow through the hollow interior portion in the longitudinal direction. The tube section 100 has a longitudinal direction, but may be bent. The size and shape of the hollow interior portion of the tube section 100 are not particularly limited as long as a liquid to be contained therein can maintain the shape of a plug in the tube section 100. Further, the size of the hollow interior portion of the tube section 100 or the shape of the cross section perpendicular to the longitudinal direction thereof may vary along the longitudinal direction of the tube section 100. Whether or not the liquid can maintain the shape of a plug in the tube section 100 depends on the conditions such as the material of the tube section 100 and the type of the liquid, and therefore, the shape of the cross section perpendicular to the longitudinal direction of the tube section 100 is designed appropriately within the scope in which the liquid can maintain the shape of a plug in the tube section 100.

The shape of the cross section perpendicular to the longitudinal direction of the external shape of the tube section 100 is also not limited. Further, the thickness (the length from the side surface of the hollow interior portion to the outer surface) of the tube section 100 is also not particularly limited. In the case where the shape of the cross section perpendicular to the longitudinal direction of the hollow interior portion of the tube section 100 is a circle, the inner diameter (the diameter of a circle of the cross section perpendicular to the longitudinal direction of the hollow interior portion) of the tube section 100 can be set to, for example, 0.5 mm or more and 3 mm or less. If the inner diameter of the tube section 100 is within this range, the plug composed of a liquid is easily formed in a wide range of the material of the tube section 100 and the type of the liquid, and therefore, such an inner diameter is preferred.

The material of the tube section 100 is not particularly limited, and for example, a glass, a polymer, a metal, or the like can be used. In particular, when a material transparent for visible light such as a glass or a polymer is selected as the material of the tube section 100, the interior portion (hollow interior) can be observed from the outside of the tube section 100, and therefore, such a material is preferred. Further, when a material through which a magnetic force is transmitted or a non-magnetic material is selected as the material of the tube section 100, in the case where magnetic particles are allowed to pass through the tube section 100 or the like, by applying a magnetic force from the outside of the tube section 100, the magnetic particles can be easily allowed to pass through the tube, and thus, such a material is more preferred.

The tube section 100 is internally provided, in the following order, with a first plug 10 composed of a first oil, a second plug 20 composed of a first washing liquid, which is phase-separated when mixed with an oil, and with which fine particles having a nucleic acid bound thereto are washed, a third plug 30 composed of a second oil, which is immiscible with the first washing liquid, a fourth plug 40 composed of an eluent, which is phase-separated when mixed with an oil, and with which the nucleic acid is eluted from the fine particles having a nucleic acid bound thereto, and a fifth plug 50 composed of a third oil, which is immiscible with the eluent.

### 1.2. First Plug, Third Plug, and Fifth Plug

The first plug 10, the third plug 30, and the fifth plug 50 are all composed of an oil. The oils of the first plug 10, the third plug 30, and the fifth plug 50 may be different types of oils or the same type of oil. As the oil, for example, one type of oil selected from silicone-based oils such as dimethyl silicone oil, paraffin-based oils, mineral oils, and mixtures thereof can be used. The liquids forming the adjacent plugs of the first plug 10, the second plug 20, the third plug 30, the fourth plug 40, and the fifth plug 50 are selected so that the adjacent plugs are not mixed with each other.

Between the first plug 10 and the third plug 30, the second plug 20 is disposed. In a region of the first plug 10 on the opposite side from the second plug 20, a plug composed of another liquid may be disposed. It is preferred that air bubbles or other liquids are not present in the first plug 10, however, air bubbles or other liquids may be present as long as the particles or the like having a nucleic acid adsorbed thereon can pass through the first plug 10. It is also preferred that air bubbles or other liquids are not present between the first plug 10 and the second plug 20, however, air bubbles or other liquids may be present as long as the particles or the like having a nucleic acid adsorbed thereon can pass through from the first plug 10 to the second plug 20. In the same manner, it is preferred that air bubbles or other liquids are not present between the second plug 20 and the third plug 30, however, air bubbles or other liquids may be present as long as the particles or the like having a nucleic acid adsorbed thereon can pass through from the second plug 20 to the third plug 30.

Between the third plug 30 and the fifth plug 50, the fourth plug 40 is disposed. In a region of the fifth plug 50 on the opposite side from the fourth plug 40, a plug composed of another liquid may be disposed. It is preferred that air bubbles or other liquids are not present in the third plug 30, however, air bubbles or other liquids may be present as long as the particles or the like having a nucleic acid adsorbed thereon can pass through the third plug 30. It is also preferred that air bubbles or other liquids are not present between the third plug 30 and the fourth plug 40, however, air bubbles or other liquids may be present as long as the particles or the like having a nucleic acid adsorbed thereon can pass through from the third plug 30 to the fourth plug 40. In the same manner, it is preferred that air bubbles or other liquids are not present between the fourth plug 40 and the fifth plug 50, however, air bubbles or other liquids may be present as long as the particles or the like having a nucleic acid adsorbed thereon can pass through from the fourth plug 40 to the fifth plug 50. Further, it is preferred that air bubbles or other liquids are not present in the fifth plug 50.

The length of each of the first plug 10, the third plug 30, and the fifth plug 50 in the longitudinal direction of the tube section 100 is not particularly limited as long as it is within a range capable of forming the plug. A specific length of each of the first plug 10, the third plug 30, and the fifth plug 50 in the longitudinal direction of the tube section 100 is 1 mm or more and 50 mm or less, and preferably 1 mm or more and 30 mm or less, more preferably 5 mm or more and 20 mm or less so that the moving distance of the particles or the like is not too long. When the length of the third plug 30 in the longitudinal direction of the tube section 100 among these plugs is made longer, in the case of adopting a configuration in which the fourth plug 40 is ejected from the end of the tube section 100 on the fifth plug 50 side, this can make it difficult to eject the second plug 20. In this case, a specific length of the third plug 30 can be set to 10 mm or more and 50 mm or less.

The first plug 10 and the fifth plug 50 each have a function of preventing substance exchange with the outside air such as evaporation or contamination from the outside of the first washing liquid (the second plug 20) and the eluent (the fourth plug 40) even if at least one end of the tube section 100 is opened. Therefore, even if at least one end of the tube section 100 is opened to the outside air, the volume of the first washing liquid or the eluent can be maintained constant, and a variation in concentration of each liquid or contamination thereof can be prevented. Due to this, the accuracy of concentration of a nucleic acid or each agent in nucleic acid extraction can be enhanced.

The third plug 30 has a function of preventing mixing of the first washing liquid (the second plug 20) and the eluent (the fourth plug 40) with each other. Further, by using an oil having a higher viscosity as the third plug 30, a “wipe-off effect” of the oil at the interface between the third plug 30 and the first washing liquid (the second plug 20) can be enhanced when the particles or the like are moved. Accordingly, when the particles or the like are moved to the third plug 30 composed of the oil from the plug composed of the first washing liquid (the second plug 20), it can make it more difficult to carry over water-soluble components adhered to the particles or the like into the third plug 30 (oil).

### 1.3. Second Plug

The second plug 20 is disposed at a position between the first plug 10 and the third plug 30 in the tube section 100. The second plug 20 is composed of the first washing liquid for washing fine particles having a nucleic acid bound thereto. The first washing liquid is a liquid immiscible with both of the oil constituting the first plug 10 and the oil constituting the third plug 30.

The first washing liquid may be any as long as it is a liquid which is phase-separated from both of the oil constituting the first plug 10 and the oil constituting the third plug 30 when mixed therewith, and examples thereof include water and buffers having a solute concentration of 10 mM or less, preferably 7 mM or less, more preferably 5 mM or less, and an acidic aqueous solution is preferred. In the case of an acidic aqueous solution, the acid to be contained is not particularly limited, however, an aqueous solution of citric acid, acetic acid, glycine hydrochloride, or the like is preferred. The first washing liquid may contain EDTA (ethylenediaminetetraacetic acid), a surfactant (such as Triton, Tween, or SLS), or the like, but is preferably a solution which does not substantially contain chaotropic substances. The pH of the solution is not particularly limited, but the lower limit of the pH is preferably 1 or more, more preferably 2 or more, further more preferably 3 or more, and most preferably 4 or more. The upper limit of the pH is preferably 6 or less, more preferably 5 or less, and most preferably 4 or less. By adopting such a first washing liquid, even if a nucleic acid or a particle having a nucleic acid bound thereto comes into contact with a solution containing an alcohol on the upstream side of the first plug, that is, even in the case where a nucleic acid is extracted with an adsorbing liquid containing an alcohol, which will be described below, or the case where particles having a nucleic acid adsorbed thereon are washed with a washing liquid containing an alcohol, the particles or the like having a nucleic acid adsorbed thereon can be efficiently washed with the first washing liquid, and also an alcohol can be prevented from being carried downstream, that is, so-called carry-over of an alcohol can be prevented.

The volume of the second plug 20 is not particularly limited, and can be appropriately set by using the amount of the particles or the like having a nucleic acid adsorbed thereon or the like as an index. For example, when the volume of the particles or the like is 0.5 μL, it is sufficient that the volume of the second plug 20 is 10 μL or more, and it is set to preferably 20 μL or more and 50 μL, or less, more preferably 20 μL, or more and 30 μL or less. If the volume of the second plug 20 is within this range, when the volume of the particles or the like is 0.5 μL, the particles or the like can be sufficiently washed. The volume of the second plug 20 is preferably larger for washing the particles or the like, but can be appropriately set in consideration of the length or diameter of the tube section 100, the length or the like of the second plug 20 in the longitudinal direction of the tube section 100 depending thereon.

The second plug 20 may be constituted by multiple plugs by being divided by an oil plug. In the case where the second plug 20 is composed of multiple plugs divided by an oil plug, multiple plugs composed of the first washing liquid are formed. Therefore, in the case where the second plug 20 is divided by an oil plug, when the substance to be washed is a water-soluble substance, the concentration of the water-soluble substance reached by the divided first washing liquid is made lower than the concentration of the water-soluble substance reached by the first washing liquid which is not divided and has the same volume, and therefore, such a configuration is preferred. The number of the divisions of the second plug 20 is an arbitrary number. However, when the substance to be washed is a water-soluble substance, for example, the second plug 20 is divided into two plugs having the same volume, the concentration of the water-soluble substance can be decreased to ¼ the concentration in the case where the second plug 20 is not divided according to calculations. The number of the divisions of the second plug 20 can be appropriately set in consideration of, for example, the length of the tube section 100, the substance to be washed, etc.

### 1.4. Fourth Plug

The fourth plug 40 is disposed at a position between the third plug 30 and the fifth plug 50 in the tube section 100 and is composed of an eluent for eluting a nucleic acid from fine particles having a nucleic acid bound thereto.

As the eluent, for example, purified water such as sterile water, distilled water, or ion exchanged water, or a buffer can be used. When water or an aqueous solution is used as the eluent, by dipping the particles or the like having a nucleic acid adsorbed thereon in an eluent, the nucleic acid adsorbed on the particles or the like can be eluted. The eluent is a liquid immiscible with both of the oil constituting the third plug 30 and the oil constituting the fifth plug 50.

The eluent may contain, in order to perform a reverse transcription reaction, a reverse transcriptase, dNTP, and a primer (oligonucleotide) for the reverse transcriptase, and may further contain, in order to perform a polymerase reaction, a DNA polymerase and a primer (oligonucleotide) for the DNA polymerase, and may also contain a probe for real-time PCR such as a TaqMan probe, a molecular beacon probe, or a cycling probe, or a fluorescent dye for an intercalator such as SYBR green. Further, the eluent preferably contains BSA (bovine serum albumin) or gelatin as a reaction inhibition inhibitor. The solvent is preferably water, and more preferably a solvent which does not substantially contain organic solvents such as ethanol and isopropyl alcohol and chaotropic substances. Further, the eluent preferably contains a salt so as to serve as a reverse transcriptase buffer and/or a DNA polymerase buffer. The salt for forming the buffer is not particularly limited as long as it does not inhibit the enzymatic reaction, but a salt such as Tris, HEPES, PIPES, or a phosphate is preferably used. The reverse transcriptase is not particularly limited, and a reverse transcriptase derived from, for example, avian myeloblast virus, Ras associated virus type 2, mouse molony murine leukemia virus, or human immunodefficiency virus type 1, or the like can be used, however, a heat-resistant enzyme is preferred. The DNA polymerase is also not particularly limited, but is preferably a heat-resistant enzyme or an enzyme for use in PCR, and there are a great number of commercially available products such as Taq polymerase, Tfi polymerase, Tth polymerase, and a modified form thereof. However, a DNA polymerase capable of performing hot start PCR is preferred.

An appropriate sequence of the primer for the DNA polymerase can be determined easily with respect to the DNA to be detected. In general, in order to amplify one type of DNA, a primer pair of a primer on the 5′ side and a primer on the 3′ side may be contained. In order to amplify multiple types of DNAs, by incorporating multiple types of primer pairs labeled with a different fluorescent dye, the device can also be applied to multiplex PCR. In this case, the number of TaqMan probes may also be appropriately increased.

The concentrations of the dNTP and the salt to be contained in the reaction mixture may be set suitable for the enzyme to be used, however, the concentration of the dNTP may be set to generally 10 to 1000 μM, preferably 100 to 500 μM, the concentration of Mg²⁺ may be set to 1 to 100 mM, preferably 5 to 10 mM, and the concentration of Cl⁻ may be set to 1 to 2000 mM, preferably 200 to 700 mM. The total ion concentration is not particularly limited, but may be higher than 50 mM, and is preferably higher than 100 mM, more preferably higher than 120 mM, further more preferably higher than 150 mM, still further more preferably higher than 200 mM. The upper limit thereof is preferably 500 mM or less, more preferably 300 mM or less, further more preferably 200 mM or less. Each oligonucleotide for the primer is used at 0.1 to 10 μM, preferably 0.1 to 1 μM. If the concentration of BSA or gelatin is 1 mg/mL or less, an inhibitory effect on reaction inhibition is small, and if the concentration thereof is 10 mg/mL or more, it may inhibit the reverse transcription reaction or the subsequent enzymatic reaction, and therefore, the concentration thereof is preferably from 1 to 10 mg/mL. In the case of using gelatin, the gelatin may be derived from, for example, cattle skin, pig skin, or cattle bone, but the origin thereof is not particularly limited thereto. If the gelatin is hardly dissolved, it may be dissolved by heating.

The volume of the fourth plug 40 composed of the eluent is not particularly limited, and can be appropriately set by using the amount of the particles or the like having a nucleic acid adsorbed thereon or the like as an index. For example, when the volume of the particles or the like is 0.5 μL, it is sufficient that the volume of the fourth plug 40 is 0.5 μL or more, and it is set to preferably 0.8 μL or more and 5 μL or less, more preferably 1 μL or more and 3 μL or less. If the volume of the eluent plug is within this range, even when the volume of the fine particles is set to 0.5 μL, the nucleic acid can be sufficiently eluted from the fine particles.

### 1.5. Structure of Nucleic Acid Extraction Device, Etc.

The nucleic acid extraction device of this embodiment includes a tube section 100, a first plug 10, a second plug 20, a third plug 30, a fourth plug 40, a fifth plug 50, and an ultrasound generating device for performing an ultrasound treatment of fine particles having a nucleic acid bound thereto, but may include a structure of adding another function. The nucleic acid extraction device of this embodiment may include a combination of the structures described below or a modification of each structure.

### 1.5.1. End Portion of Tube Section

FIG. 2 is a view schematically showing a nucleic acid extraction device 1010 which is a modification of the nucleic acid extraction device. The nucleic acid extraction device of this embodiment may be, for example, configured such that the end of the tube section 100 on the fifth plug 50 side is opened. That is, as shown in FIG. 2, in the nucleic acid extraction device 1010, the end of the tube section 100 on the fifth plug 50 side is in an opened state. According to the nucleic acid extraction device 1010, it is possible to sequentially eject the fifth plug 50 and the fourth plug 40 by applying pressure to the inside of the tube section 100 from the first plug 10 side of the tube section 100. Accordingly, by using the nucleic acid extraction device 1010, the eluate (the fourth plug 40) containing a target nucleic acid can be easily dispensed into, for example, a reaction vessel or the like for PCR.

### 1.5.2. Stopper

FIG. 3 is a view schematically showing a nucleic acid extraction device 1020 which is a modification of the nucleic acid extraction device. As shown in FIG. 3, the nucleic acid extraction device of this embodiment may further include a detachable stopper 110 which seals, for example, the end of the tube section 100 on the fifth plug 50 side. The stopper 110 can be formed from, for example, a rubber, an elastomer, a polymer, or the like. In the case where the tube section 100 is sealed with the stopper 110, the stopper 110 may be in contact with the fifth plug 50, or a gas such as air may be disposed between the fifth plug 50 and the stopper 110. The stopper 110 is freely detachable, but its mechanism is not particularly limited. In the example shown in FIG. 3, a configuration in which the stopper 110 is fixed by inserting a part thereof in the inside of the tube section 100 is shown, however, the stopper 110 may be in the form of a cap.

In the nucleic acid extraction device 1020, in the case where the stopper 110 is detached, the end of the tube section 100 on the fifth plug 50 side is opened so that the device 1020 has the same configuration as the nucleic acid extraction device 1010 shown in FIG. 2 described above, and by using the nucleic acid extraction device 1020, the eluate (the fourth plug 40) containing a target nucleic acid can be easily dispensed into, for example, a reaction vessel or the like for PCR. Further, in the case where the device 1020 is in a state where the end of the tube section 100 on the fifth plug 50 side is sealed with the stopper 110 (a state shown in FIG. 3), an effect of preventing each plug from moving in the tube section 100 is obtained. Accordingly, for example, in the case where the particles or the like are moved in the tube section 100, the plugs can be prevented from moving accompanying the movement of the particles or the like.

### 1.5.3. Vessel

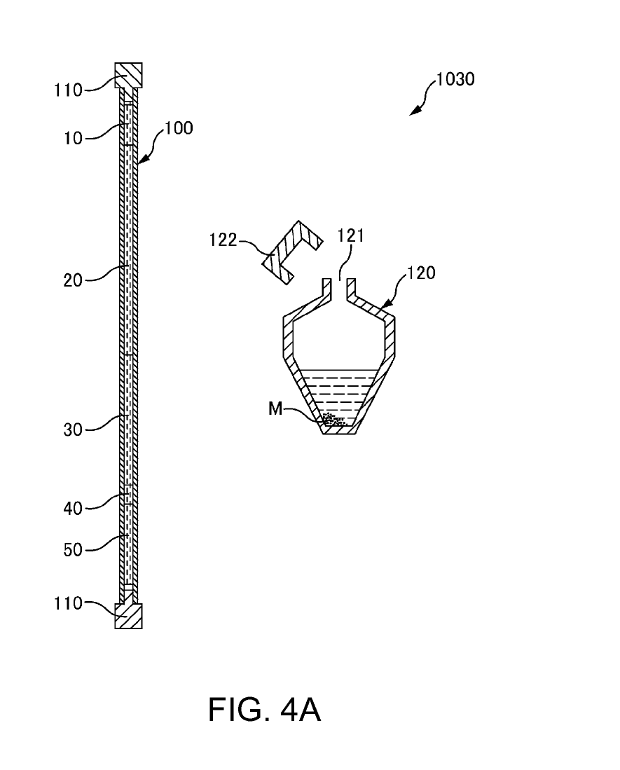

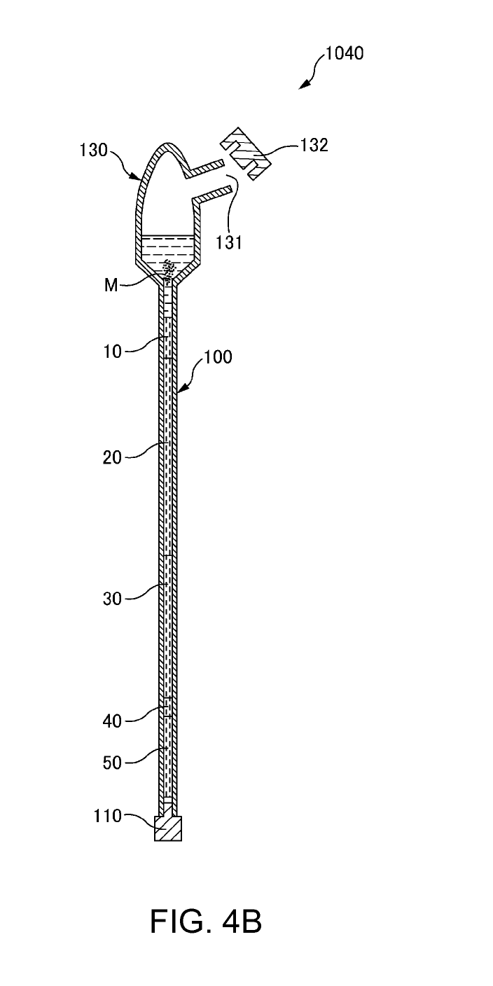

FIG. 4A is a view schematically showing a nucleic acid extraction device 1030 which is one example of the structure of the nucleic acid extraction device. As illustrated in FIG. 4A, the nucleic acid extraction device 1030 further includes a detachable vessel 120 which can be connected to the upstream end of the tube section 100 on the first plug 10 side so that the insides thereof communicate with each other.

The vessel 120 can be formed as an independent member. The vessel 120 can contain a liquid therein, and has an opening 121 through which a liquid or a solid can be introduced or discharged. In the example shown in FIG. 4A, the opening 121 of the vessel 120 is configured to be connected to the end of the tube section 100 on the first plug 10 side so that the insides thereof communicate with each other. Further, the vessel 120 may have multiple openings 121 and one of the openings 121 in this case may be configured to be connected to the end of the tube section 100 on the first plug 10 side so that the insides thereof communicate with each other.

The internal volume of the vessel 120 is not particularly limited, but can be set to, for example, 0.1 mL or more and 100 mL or less. The opening 121 of the vessel 120 may have a structure capable of being sealed with a cap 122 as needed. The material of the vessel 120 is not particularly limited, but for example, a polymer, a metal, or the like can be used.

The opening 121 of the vessel 120 can be connected to the end of the tube section 100 on the first plug 10 side, however, the way of connection between the vessel 120 and the tube section 100 is not particularly limited as long as the contents do not leak. In the case where the vessel 120 and the tube section 100 are connected to each other, the inside of the vessel 120 and the inside of the tube section 100 can communicate with each other. Further, the vessel 120 can be detached from the tube section 100 as needed.

By providing the vessel 120 as in the nucleic acid extraction device 1030, in the vessel 120, for example, the particles or the like, the adsorbing liquid, and a sample are placed, and a nucleic acid can be adsorbed on the particles or the like. Thereafter, by connecting the vessel 120 to the end of the tube section 100 on the first plug 10 side, the device can be brought to a state where the particles or the like can be easily introduced into the tube section 100 from the first plug 10 side of the tube section 100.

The adsorbing liquid refers to a liquid which is used for adsorbing a nucleic acid on fine particles (for example, magnetic particles M) serving as a nucleic acid-binding solid-phase carrier. The adsorbing liquid is not particularly limited as long as it contains a chaotropic substance, but a surfactant may be contained therein for the purpose of disrupting cell membranes or denaturing proteins contained in cells. This surfactant is not particularly limited as long as it is generally used for extracting nucleic acids from cells or the like. Specific examples thereof include nonionic surfactants such as Triton surfactants (such as Triton-X) and Tween surfactants (such as Tween 20) and anionic surfactants such as sodium n-lauroylsarcosinate (SLS). However, particularly, it is preferred to use a nonionic surfactant in an amount ranging from 0.1 to 2%. Further, the adsorbing liquid preferably contains a reducing agent such as 2-mercaptoethanol or dithiothreitol. The adsorbing liquid may be a buffer, but preferably has a neutral pH ranging from 6 to 8. In view of this, specifically, the adsorbing liquid preferably contains a guanidine salt (3 to 7 M), a nonionic surfactant (0 to 5%), EDTA (0 to 0.2 mM), a reducing agent (0 to 0.2 M), etc.

The chaotropic substance is not particularly limited as long as it generates a chaotropic ion (a monovalent anion having a large ionic radius) in an aqueous solution, has an activity to increase the water solubility of a hydrophobic molecule, and contributes to the adsorption of a nucleic acid on the fine particles. Specific examples thereof include guanidine thiocyanate, guanidine hydrochloride, sodium iodide, potassium iodide, and sodium perchlorate. Among these, guanidine thiocyanate or guanidine hydrochloride having a high protein denaturation activity is preferred. The concentration of such a chaotropic substance varies depending on the respective substances, and for example, when guanidine thiocyanate is used, the concentration thereof is preferably in the range of 3 to 5.5 M, and when guanidine hydrochloride is used, the concentration thereof is preferably 5 M or more.

The adsorbing liquid preferably contains an alcohol or acetonitrile. In this case, the concentration of the alcohol or the like is not particularly limited, however, the lower limit thereof may be 10% or more, or may be 20% or more, or may be 30% or more, but most preferably 40% or more. The upper limit thereof may be 80% or less, or may be 70% or less, or may be 60% or less, but most preferably 50% or less. The type of the alcohol is not particularly limited, but examples thereof include methanol, ethanol, and propanol. By adding an alcohol or the like to the adsorbing liquid, an effect of adsorbing a nucleic acid on the particles or the like is enhanced, and thus, the efficiency of extraction of a nucleic acid can be enhanced when it is used in the nucleic acid extraction device.

The vessel 120 can be shaken in a state where it is not connected to the tube section 100, and therefore, a liquid in the vessel 120 can be sufficiently stirred. Accordingly, a nucleic acid can be rapidly adsorbed on the particles or the like. The vessel 120 may have a cap 122 which seals the opening 121. Further, by appropriately changing the amount of a sample to be introduced into the vessel 120 and the volume of the liquid (particularly, the fourth plug 40) in the tube section 100, a nucleic acid in the sample can also be quantitatively concentrated in the eluate in the fourth plug 40.

In the case where a flexible material such as a rubber, an elastomer, or a polymer is selected as the material of the vessel 120, pressure can be applied to the inside of the tube section 100 by deforming the vessel 120 in a state where the vessel 120 is connected to the tube section 100. According to this, when the eluate in the fourth plug 40 is ejected from the end of the tube section 100 on the fifth plug 50 side, pressure can be easily applied from the first plug 10 side of the tube section 100. As a result, the eluate can be easily dispensed into, for example, a reaction vessel or the like for PCR.

### 1.5.4. Liquid Reservoir Section

FIG. 4B is a view schematically showing a nucleic acid extraction device 1040 which is one example of the structure of the nucleic acid extraction device. As illustrated in FIG. 4B, in the nucleic acid extraction device 1040, a liquid reservoir section 130 which communicates with the tube section 100 is formed at the end of the tube section 100 on the first plug 10 side. The inside of liquid reservoir section 130 and the inside of the tube section 100 communicate with each other.

The liquid reservoir section 130 can contain a liquid therein. The liquid reservoir section 130 has an opening 131 capable of introducing a substance into the liquid reservoir section 130 from the outside. The position where the opening 131 is formed in the liquid reservoir section 130 is not particularly limited. The liquid reservoir section 130 may have multiple openings 131. The internal volume of the liquid reservoir section 130 is not particularly limited, but can be set to, for example, 0.1 mL or more and 100 mL or less. The material of the liquid reservoir section 130 is not particularly limited, but for example, a polymer, a metal, or the like can be used, and may be the same material as that of the tube section 100.

By providing the liquid reservoir section 130 as in the nucleic acid extraction device 1040, in the liquid reservoir section 130, for example, the particles or the like, the adsorbing liquid, and a sample are placed, and a nucleic acid can be adsorbed on the particles or the like. Then, the particles or the like can be easily introduced into the tube section 100 from the first plug 10 side of the tube section 100.

Further, the liquid reservoir section 130 can be shaken along with the tube section 100, and therefore, a liquid in the liquid reservoir section 130 can be sufficiently stirred. Accordingly, a nucleic acid can be rapidly adsorbed on the particles or the like. In addition, by appropriately changing the amount of a sample to be introduced into the liquid reservoir section 130 and the volume of the liquid in the tube section 100, a nucleic acid in the sample can be quantitatively concentrated in the eluate.

In the case where the liquid reservoir section 130 is provided as in the nucleic acid extraction device 1040, a detachable cap 132 which seals the opening 131 of the liquid reservoir section 130 may further be provided. Then, when a flexible material such as a rubber, an elastomer, or a polymer is selected as the material of the liquid reservoir section 130, pressure can be applied to the inside of the tube section 100 by deforming the liquid reservoir section 130 in a state where the cap 132 is attached to the liquid reservoir section 130.

According to this, when the eluate in the fourth plug 40 in which a nucleic acid has been eluted is ejected from the end of the tube section 100 on the fifth plug 50 side, pressure can be easily applied from the first plug 10 side of the tube section 100. Accordingly, a step of introducing a sample into the liquid reservoir section 130 to a step of easily dispensing the eluate into, for example, a reaction vessel or the like for PCR can be performed. Further, by attaching the cap 132, liquid leakage can be prevented when shaking the liquid reservoir section 130 along with the tube section 100, and therefore, the efficiency of adsorption of a nucleic acid on the particles or the like can be further improved.

### 1.5.5. Sixth Plug and Seventh Plug

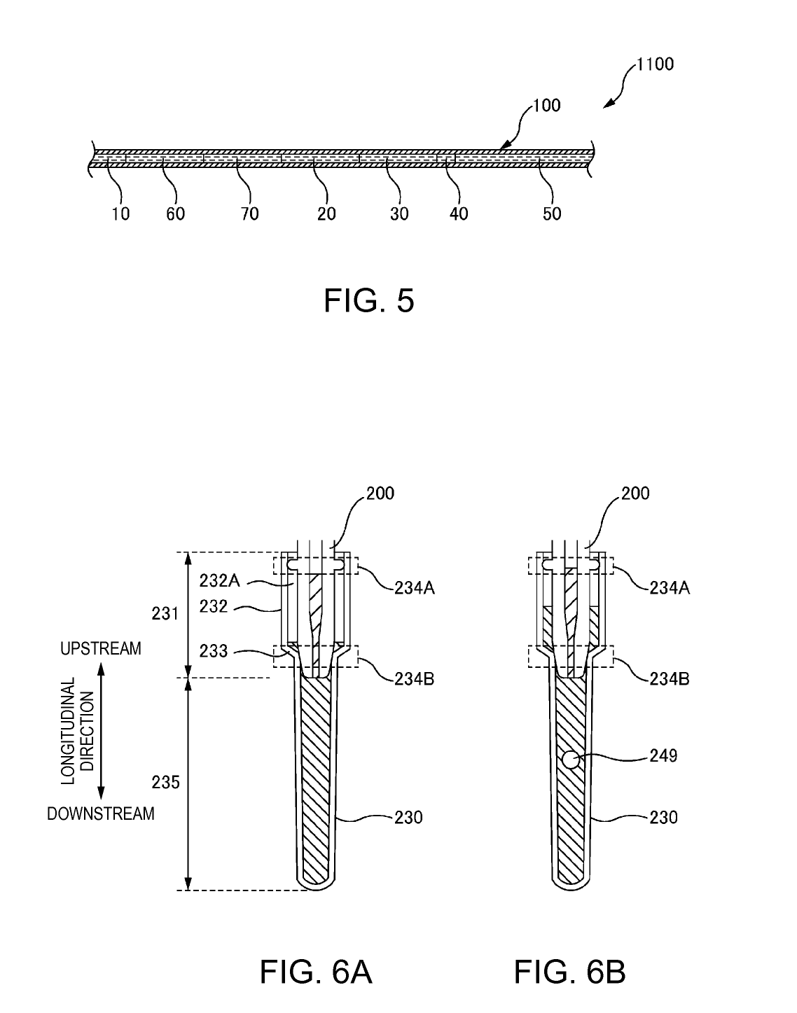

The nucleic acid extraction device of this embodiment may include a sixth plug and a seventh plug in the tube section. FIG. 5 is a view schematically showing a nucleic acid extraction device 1100 including a sixth plug 60 and a seventh plug 70 in a tube section 100.

The nucleic acid extraction device 1100 is configured to add a sixth plug 60 composed of a second washing liquid which is phase-separated when mixed with an oil, and with which fine particles having a nucleic acid bound thereto are washed, and a seventh plug 70 composed of a fourth oil in this order from the first plug 10 side between the first plug 10 and the second plug 20 in the tube section 100 of the above-described nucleic acid extraction device.

The sixth plug 60 is composed of a second washing liquid. The second washing liquid may be any as long as it is a liquid which is phase-separated from both of the oil constituting the first plug 10 and the oil constituting the seventh plug 70 when mixed therewith. The second washing liquid is preferably water or an aqueous solution with a low salt concentration. In the case of the aqueous solution with a low salt concentration, a buffer is preferred. The salt concentration in the aqueous solution with a low salt concentration is preferably 100 mM or less, more preferably 50 mM or less, and most preferably 10 mM or less. The lower limit of the salt concentration in the aqueous solution with a low salt concentration is not particularly limited, but is preferably 0.1 mM or more, more preferably 0.5 mM or more, and most preferably 1 mM or more. This solution may contain a surfactant such as Triton, Tween, or SLS, and the pH of the solution is not particularly limited. The salt for forming the buffer is not particularly limited, but a salt such as Tris, HEPES, PIPES, or a phosphate is preferably used. It does not matter if the solution contains a chaotropic substance.

It is preferred that this washing liquid contains an alcohol for enhancing the washing effect. In particular, in order to enhance the effect of using an acidic solution as the first washing liquid, it is preferred that at least one of the adsorbing liquid and the second washing liquid contains an alcohol. In this case, the concentration of the alcohol is not particularly limited, however, the lower limit thereof may be 50% or more, or may be 60% or more, but most preferably 70% or more. The upper limit of the concentration of the alcohol may be 90% or less, or may be 80% or less, but most preferably 70% or less. The type of the alcohol is not particularly limited, but examples thereof include methanol, ethanol, propanol and acetonitrile. In terms of washing a nucleic acid and the particles or the like having a nucleic acid adsorbed thereon, the washing effect can be enhanced by incorporating an alcohol in at least one of the above-described adsorbing liquid and the second washing liquid, but it is preferred that both of the adsorbing liquid and the second washing liquid contain an alcohol because the washing effect can be further enhanced.

The volume of the sixth plug 60 is not particularly limited, and can be appropriately set by using the amount of the particles or the like having a nucleic acid adsorbed thereon or the like as an index. For example, when the volume of the particles or the like is 0.5 μL, it is sufficient that the volume of the sixth plug 60 is 10 μL or more, and it is set to preferably 20 μL or more and 50 μL, or less, more preferably 20 μL, or more and 30 μL, or less. If the volume of the sixth plug 60 is within this range, when the volume of the particles or the like is set to 0.5 μL, the particles or the like can be sufficiently washed. The volume of the sixth plug 60 is preferably larger for washing the particles or the like, but can be appropriately set in consideration of the length or diameter of the tube section 100, the length or the like of the sixth plug 60 in the longitudinal direction of the tube section 100 depending thereon.

The sixth plug 60 may be constituted by multiple plugs by being divided by an oil plug. In the case where the sixth plug 60 is composed of multiple plugs divided by an oil plug, the compositions of the washing liquids in the respective plugs may be either the same or different, and are not particularly limited, but preferably contain an alcohol as described above.

The seventh plug 70 is composed of an oil immiscible with the liquids of the adjacent sixth plug 60 and second plug 20. The oil of the seventh plug 70 and the oil of the first plug 10, the third plug 30, or the fifth plug 50 may be the same type of oil or different types of oils. Examples of the oil can include those listed as examples of the oil of the first plug 10 or the like.

It is preferred that air bubbles or other liquids are not present in the seventh plug 70, however, air bubbles or other liquids may be present as long as the particles or the like having a nucleic acid adsorbed thereon can pass through the seventh plug 70. It is also preferred that air bubbles or other liquids are not present between the seventh plug 70 and the adjacent second plug 20 and between the seventh plug 70 and the adjacent sixth plug 60, however, air bubbles or other liquids may be present as long as the particles or the like having a nucleic acid adsorbed thereon can move in the tube section 100.

The length of the seventh plug 70 in the longitudinal direction of the tube section 100 is not particularly limited as long as it is within a range capable of forming the plug. A specific length of the seventh plug 70 in the longitudinal direction of the tube section 100 is 1 mm or more and 50 mm or less, and it is preferably 1 mm or more and 30 mm or less, more preferably 5 mm or more and 20 mm or less so that the moving distance of the particles or the like is not too long.

The seventh plug 70 has a function of preventing mixing of the second washing liquid (the sixth plug 60) and the first washing liquid (the second plug 20) with each other. Further, by using an oil having a higher viscosity as the seventh plug 70, a “wipe-off effect” of the oil at the interface between the seventh plug 70 and the second washing liquid (the sixth plug 60) can be enhanced when the particles or the like are moved. Accordingly, when the particles or the like are moved to the seventh plug 70 composed of the oil from the plug composed of the second washing liquid (the sixth plug 60), it can make it more difficult to carry over water-soluble components adhered to the particles or the like into the seventh plug 70.

According to the nucleic acid extraction device 1100, the particles or the like having a nucleic acid adsorbed thereon can be washed in the sixth plug 60 as well as the second plug 20. Accordingly, the efficiency of washing of the particles or the like can be further enhanced.

In the nucleic acid extraction device 1100, a chaotropic agent may be incorporated in the second washing liquid of the sixth plug 60. For example, when guanidine hydrochloride is incorporated in the second washing liquid, it is possible to wash the particles or the like in the sixth plug 60 while maintaining or enhancing the adsorption of a nucleic acid adsorbed on the particles or the like. The concentration of guanidine hydrochloride in the case where guanidine hydrochloride is incorporated in the sixth plug 60 can be set to, for example, 3 mol/L or more and 10 mol/L or less, preferably 5 mol/L or more and 8 mol/L or less. If the concentration of guanidine hydrochloride is within this range, other foreign substances and the like can be washed off while more stably adsorbing a nucleic acid adsorbed on the particles or the like.

Then, as described above, as the first washing liquid of the second plug 20, an acidic solution is used, an alcohol adsorbed on the particles or the like in the sixth plug 60 can be efficiently washed off in the second plug 20, and thus, carry-over of an alcohol to the eluent, or the reverse transcription reaction system or the nucleic acid amplification reaction system thereafter can be reduced.

It is easily understood that also in the case of the nucleic acid extraction device 1100 including the sixth plug 60 and the seventh plug 70, in the tube section 100, the above-described stopper, vessel, liquid reservoir section, etc. can be added to the structure, and the same effect as described above can be obtained.

### 1.5.6. Nucleic Acid Amplification Reaction Vessel

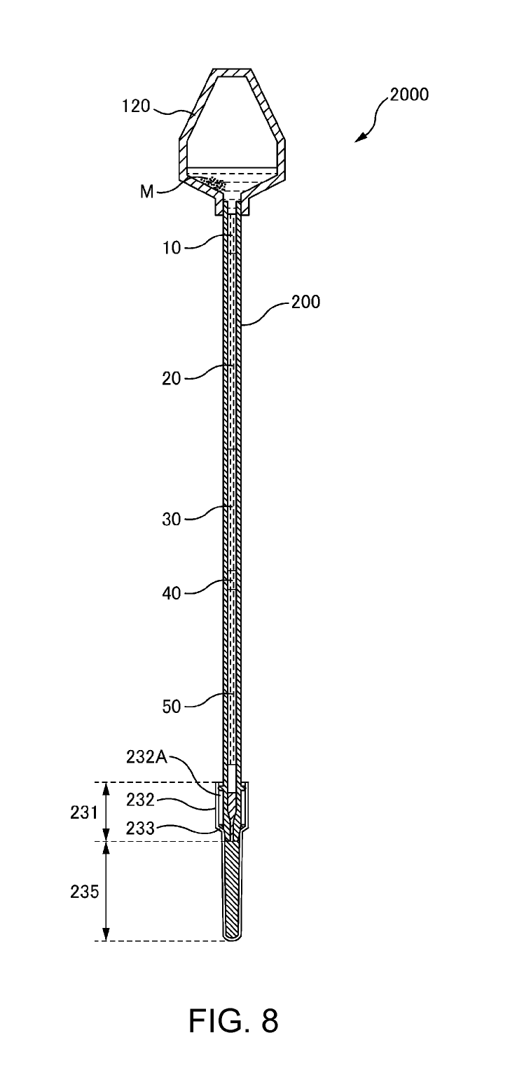

The nucleic acid extraction device according to the invention may be configured as a nucleic acid amplification reaction cartridge including a nucleic acid amplification reaction vessel, which communicates with the downstream end of a tube and contains an oil. FIGS. 6A and 6B show one example of a structure of the nucleic acid amplification reaction vessel, and FIG. 8 shows one example of an overall structure of a nucleic acid amplification reaction cartridge.

FIG. 6A is an explanatory view of an initial state. FIG. 6B is an explanatory view of a state after an eluate is pushed out from a tube 200. A nucleic acid amplification reaction vessel 230 serves as a vessel for receiving a liquid pushed out from the tube 200 and also serves as a vessel for storing an eluate 249 in a thermal cycling process.

The nucleic acid amplification reaction vessel 230 has a seal forming section 231 and a flow channel forming section 235. The seal forming section 231 is apart into which the tube 200 is inserted, and prevents an oil overflowing from the flow channel forming section 235 from leaking to the outside. The flow channel forming section 235 is apart on the downstream side of the seal forming section 231, and forms a flow channel in which the eluate 249 in the form of a liquid droplet moves. The nucleic acid amplification reaction vessel 230 is fixed to the tube 200 at the following two sites: an upper seal section 234A and a lower seal section 234B of the seal forming section 231.

The seal forming section 231 has an oil receiving section 232 and a stepped section 233.

The oil receiving section 232 is a cylindrical part and functions as a reservoir which receives an oil overflowing from the flow channel forming section 235. There is a gap between the inner wall of the oil receiving section 232 and the outer wall of the tube 200, and this gap serves as an oil receiving space 232A which receives an oil overflowing from the flow channel forming section 235.

The upper seal section 234A is formed by contact between the inner wall on the upstream side of the oil receiving section 232 and an annular protruding portion of the tube 200. The upper seal section 234A is a seal which prevents an oil in the oil receiving space 232A from leaking to the outside while allowing air to pass therethrough. The upper seal section 234A is provided with a vent to such an extent that an oil does not leak due to the surface tension of the oil. The vent of the upper seal section 234A may be a gap between the protruding portion of the tube 200 and the inner wall of the oil receiving section 232, or may be a hole, a groove, or a notch formed in the protruding portion of the tube 200. In addition, the upper seal section 234A may be formed from an oil absorbing material which absorbs an oil.

The stepped section 233 is a part with a step provided on the downstream side of the oil receiving section 232. The inner diameter of the downstream portion of the stepped section 233 is smaller than the inner diameter of the oil receiving section 232. The inner wall of the stepped section 233 is in contact with the outer wall on the downstream side of the tube 200. The lower seal section 234B is formed by the contact between the inner wall of the stepped section 233 and the outer wall on the downstream side of the tube 200. The lower seal section 234B is a seal part which allows an oil in the flow channel forming section 235 to flow in the oil receiving space 232A, but also resists the flow. Due to the pressure loss in the lower seal section 234B, the pressure in the flow channel forming section 235 becomes higher than the outside pressure, and therefore, even if the liquid in the flow channel forming section 235 is heated in the thermal cycling process, air bubbles are hardly generated in the liquid in the flow channel forming section 235.

The flow channel forming section 235 is a tubular part and serves as a vessel for forming a flow channel in which the eluate 249 in the form of a liquid droplet moves. The flow channel forming section 235 is filled with an oil. The flow channel forming section 235 on the upstream side is closed by the end of the tube 200, and the end of the tube 200 is opened toward the flow channel forming section 235. The inner diameter of the flow channel forming section 235 is larger than the inner diameter of the tube 200, and also larger than the outer diameter of the eluate 249 when it is in the form of a sphere. The inner wall of the flow channel forming section 235 preferably has water repellency to such an extent that the water-soluble eluate 249 is not adhered thereto.

As shown in FIG. 6A, in the initial state, the flow channel forming section 235 of the nucleic acid amplification reaction vessel 230 is filled with an oil. The interface of the oil is located on a relatively downstream side of the oil receiving space 232A.

As shown in FIG. 6B, even if the eluate 249 is pushed out from the tube 20, a gas does not flow into the flow channel forming section 235 because the flow channel forming section 235 is filled with an oil in advance.

Before the eluate 249 is pushed out from the tube 200, first, an oil in the oil plug on the most downstream side of the tube 200 flows into the flow channel forming section 235, and then, the oil flows into the oil receiving space 232A from the flow channel forming section 235 in an amount equivalent to that of the oil flowing into the flow channel forming section 235, and thus, the interface of the oil in the oil receiving space 232A moves up. At this time, due to the pressure loss in the lower seal section 234B, the pressure of the liquid in the flow channel forming section 235 is increased. After the oil plug on the most downstream side is pushed out from the tube 200, the eluate 249 flows into the flow channel forming section 235 from the tube 200. In the case where the eluate 249 contains a nucleic acid to be amplified and a reagent for performing a nucleic acid amplification reaction, the eluate 249 flowing thereinto is shaped in the form of a liquid droplet and is directly used in PCR.

Such an integrated nucleic acid amplification reaction cartridge can be favorably used in, for example, a rotary PCR apparatus as the apparatus whose principle is disclosed in JP-A-2012-115208.

### 1.5.7. Ultrasound Generating Device

The ultrasound generating device is not particularly limited, and may be any as long as it can perform an ultrasound treatment of the eluate ejected from the tube or the eluate in the tube. For example, a commercially available ultrasound generating device can be used. The position where the device is placed is also not particularly limited.

### 2. NUCLEIC ACID EXTRACTION KIT

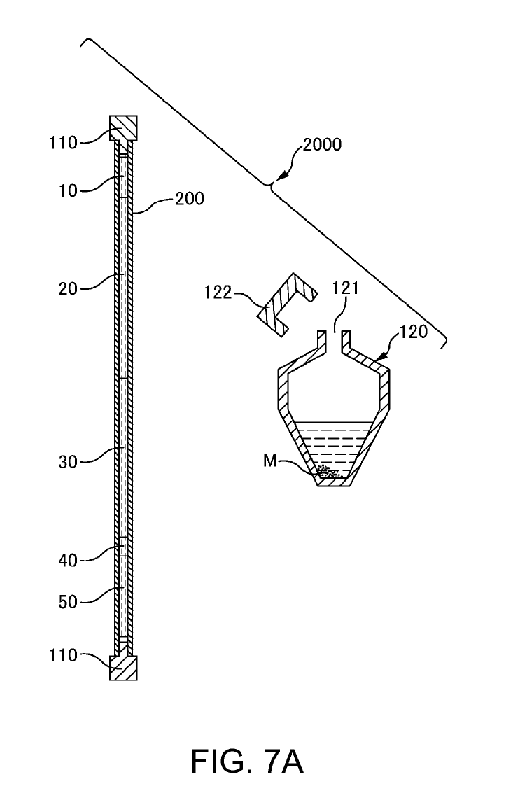

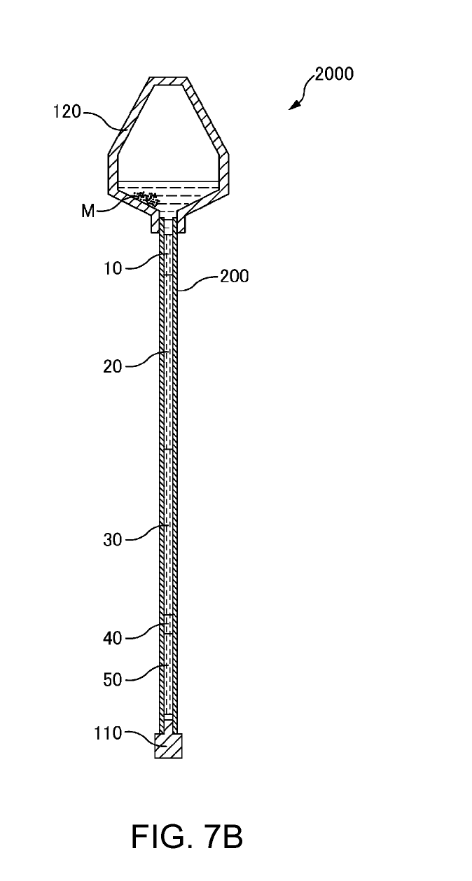

FIG. 7A is a schematic view showing one example of the nucleic acid extraction kit of this embodiment, A nucleic acid extraction kit 2000 illustrated in FIG. 7A includes components constituting the principal part of the nucleic acid extraction device described above. The same components as those described in the section “1. Nucleic Acid Extraction Device” are denoted by the same reference numerals, and a detailed description thereof will be omitted.

The nucleic acid extraction kit 2000 of this embodiment includes a tube 200, in which a first plug 10 composed of an oil, a second plug 20 composed of a first washing liquid immiscible with an oil, a third plug 30 composed of an oil, a fourth plug 40 composed of an eluent immiscible with an oil, and a fifth plug 50 composed of an oil are internally disposed in this order, an ultrasound generating device which performs an ultrasound treatment of fine particles having a nucleic acid bound thereto, and a vessel 120 which can be connected to the end of the tube 200 on the first plug 10 side so that the insides thereof communicate with each other.

The tube 200 has a configuration such that both ends of the tube section 100 of the nucleic acid extraction device 1000 are opened, has a hollow interior portion, and has a cylindrical shape capable of allowing a liquid to flow through the hollow interior portion in the longitudinal direction. The internal shape, external shape, size, properties, material, etc. of the tube 200 are the same as those of the tube section 100 of the nucleic acid extraction device 1000. The plugs disposed in the inside of the tube 200 are the same as the plugs disposed in the tube section 100 of the nucleic acid extraction device 1000. Further, both ends of the tube 200 may be sealed with a detachable stopper 110. In the case where both ends of the tube 200 are sealed with the stopper 110, for example, the storage and transport of the nucleic acid extraction kit 2000 are further facilitated. In the case where the end of the tube 200 on the fifth plug 50 side is sealed with the stopper 110 at the time of using the tube 200, when the particles or the like are moved in the tube 200, the respective plugs can be prevented from moving in the tube 200, and therefore, washing and extraction can be further facilitated. Moreover, since the stopper 110 is detachable, the end of the tube 200 on the fifth plug 50 side can be opened, and therefore, the eluate in the fourth plug 40 in which a nucleic acid has been eluted is easily ejected from the end of the tube 200 on the fifth plug 50 side.

The vessel 120 and the ultrasound generating device are the same as the vessel 120 and the ultrasound generating device described in the section about the nucleic acid extraction device 1000.

In the example shown in FIG. 7A, both ends of the tube 200 are sealed with the detachable stopper 110. The nucleic acid extraction kit 2000 may include a cap 122 which detachably seals an opening 121 of the vessel 120, and the opening 121 of the vessel 120 may be sealed with the detachable cap 122. Further, in the nucleic acid extraction kit 2000, some or all of the components of the adsorbing liquid may be placed in the vessel 120.

In the nucleic acid extraction kit 2000, the adsorbing liquid and magnetic particles may be placed in the vessel 120. By doing this, when a sample is introduced into the vessel 120, a step of adsorbing a nucleic acid contained in the sample on the magnetic particles can be performed in the vessel 120. Accordingly, it is not necessary to prepare another vessel, and further, a pretreatment for PCR can be performed more rapidly. Further, in this case, the opening 121 of the vessel 120 may be sealed by the detachable cap 122 as needed. The magnetic particles will be described in detail below.

Further, as the material of the vessel 120, when a flexible material is used as described above, pressure can be applied to the inside of the tube 200 by deforming the vessel 120 in a state where the vessel 120 is connected to the tube 200. According to this, when the eluate in the fourth plug 40 in which a nucleic acid has been eluted is ejected from the end of the tube 200 on the fifth plug 50 side, pressure can be easily applied from the first plug 10 side of the tube 200. As a result, the eluate can be easily dispensed into, for example, a reaction vessel or the like for PCR.

In the nucleic acid extraction kit 2000, other than the tube 200 and the vessel 120, components, for example, a stopper, a cap, an instruction manual, a reagent, a case, and the like may be contained. Further, here, an example in which five plugs are disposed in the tube 200 is described, however, it is easily understood that in the same manner as described in the section “1.6. Nucleic Acid Extraction Device”, a sixth plug 60, a seventh plug 70, or the like, or another plug according to need may be disposed in the tube 200 (tube section 100).

Since the nucleic acid extraction kit 2000 of this embodiment includes the vessel 120 which can be connected to the end of the tube 200 on the first plug 10 side so that the insides thereof communicate with each other, by placing the particles or the like and a sample in the vessel 120, a nucleic acid can be adsorbed on the particles or the like, and by connecting the vessel 120 to the end of the tube 200 on the first plug 10 side, the particles or the like can be easily introduced into the tube 200 from the first plug 10 side of the tube 200. Further, since the nucleic acid extraction kit 2000 of this embodiment includes the vessel 120, the vessel 120 can be shaken, and thus, a liquid in the vessel 120 can be sufficiently stirred. Accordingly, a nucleic acid can be rapidly adsorbed on the particles or the like.

Further, by connecting the vessel 120 to the tube 200, the particles or the like having a nucleic acid adsorbed thereon is easily moved to the fourth plug 40 by being introduced into the tube 200 from the end on the first plug 10 side. By doing this, the extraction of a nucleic acid can be easily performed in an extremely short time. In the nucleic acid extraction kit 2000, by moving the particles or the like having a nucleic acid adsorbed thereon in the tube 200, an eluate containing a nucleic acid in high purity can be obtained. Therefore, according to the nucleic acid extraction kit 2000, the time and labor required for a pretreatment for PCR can be largely reduced.

### 3. NUCLEIC ACID EXTRACTION METHOD

All of the above-described nucleic acid extraction device, nucleic acid amplification reaction cartridge, nucleic acid extraction kit, and modifications thereof can be favorably used for the nucleic acid extraction method of this embodiment. Hereinafter, as one example of the nucleic acid extraction method of this embodiment, a method using the above-described nucleic acid extraction kit 2000 will be described.

The nucleic acid extraction method of this embodiment includes a step of introducing a sample containing a nucleic acid into a vessel 120 which has flexibility and contains fine particles such as magnetic particles M and an adsorbing liquid, a step of adsorbing the nucleic acid on the magnetic particles M by shaking the vessel 120, a step of connecting the vessel 120 to the end of a tube 200 on a first plug 10 side, in which a first plug 10 composed of an oil, a second plug 20 composed of a first washing liquid immiscible with an oil, a third plug 30 composed of an oil, a fourth plug 40 composed of an eluent immiscible with an oil, and a fifth plug 50 composed of an oil are internally disposed in this order, so that the inside of the vessel 120 and the inside of the tube 200 communicate with each other, a step of moving the magnetic particles M from the inside of the vessel 120 to the position of the fourth plug 40 by passing the magnetic particles M through the inside of the tube 200 by applying a magnetic force, and a step of eluting the nucleic acid adsorbed on the fine particles in an eluate by washing the fine particles having the nucleic acid adsorbed thereon with a first washing liquid, followed by an ultrasound treatment of the fine particles having the nucleic acid adsorbed thereon.

In the nucleic acid extraction method of this embodiment, various types of particles (such as silica particles, polymer particles, and magnetic particles) can be used as long as the particles can adsorb a nucleic acid using an adsorbing liquid and can move in the tube 200, however, in an embodiment of the nucleic acid extraction method described below, magnetic particles M which contain a magnetic body and can adsorb a nucleic acid on the surface thereof are used. In the case where particles or the like other than the magnetic particles M are moved in the tube, for example, the particles or the like can be moved by utilizing a gravitational force or a potential difference.

In the nucleic acid extraction method of this embodiment, a material through which a magnetic force is transmitted is selected as the material of the vessel 120 and the tube 200, and by applying a magnetic force from the outside of the vessel 120 and the tube 200, the magnetic particles M are moved in the vessel 120 and the tube 200.

In the sample, a nucleic acid to be a target is contained. Hereinafter this is sometimes simply referred to as “target nucleic acid”. The target nucleic acid is extracted from the sample and eluted in an eluate by the nucleic acid extraction method of this embodiment. Thereafter, for example, in the case where the nucleic acid is an mRNA, the nucleic acid is reverse-transcribed into a cDNA and is used as a template for PCR. In the case where the nucleic acid is a cDNA or a genomic DNA, the nucleic acid is directly used as a template for PCR. As the sample, blood, nasal mucus, oral mucosa, various types of biological samples, and other than these, a partially purified nucleic acid solution or the like can be used.

### 3.1. Step of Introducing Sample in Vessel

The step of introducing the sample into the vessel 120 can be performed by, for example, adhering the sample to a cotton swab, inserting the cotton swab from the opening 121 of the vessel 120, and dipping the cotton swab in the adsorbing liquid. The sample may also be introduced from the opening 121 of the vessel 120 using a pipette or the like. In the case where the sample is in the form of a paste or a solid, for example, the sample may be adhered to the inner wall of the vessel 120 or thrown into the vessel 120 from the opening 121 of the vessel 120 using a spoon, forceps, or the like.

### 3.2. Step of Adsorbing Nucleic Acid on Magnetic Particles

The step of adsorbing the nucleic acid is performed by shaking the vessel 120. When there is a cap 122 which seals the opening 121 of the vessel 120, this step can be efficiently performed by sealing the vessel 120 using this cap 122. By this step, the target nucleic acid is adsorbed on the surfaces of the magnetic particles M by the action of a chaotropic agent. In this step, nucleic acids other than the target nucleic acid, or proteins may be adsorbed on the surfaces of the magnetic particles M in addition to the target nucleic acid.

As the method of shaking the vessel 120, a device such as a vortex shaker may be used, or an operator may shake the vessel by hand. Further, by utilizing the magnetism of the magnetic particles M, the vessel 120 may be shaken while applying a magnetic field from the outside. The time of shaking the vessel 120 can be appropriately set, however, for example, in the case where the approximate shape of the vessel 120 is a cylinder having a diameter of about 20 mm and a height of about 30 mm, by merely shaking the vessel 120 with hand for about 10 seconds, the contents in the vessel 120 can be sufficiently stirred and the nucleic acid is adsorbed on the surfaces of the magnetic particles M.

### 3.3 Step of Connecting Vessel to Tube

Subsequently, as shown in FIG. 7B, the vessel 120 is connected to the end of the tube 200 on the first plug 10 side. Even when the stopper 110 on the first plug 10 side is detached, the respective plugs in the tube 200 hardly move in the tube 200 because the stopper 110 on the fifth plug 50 side is attached to the tube. In the case where the stopper 110 is attached to the end of the tube 200 on the first plug 10 side, this step is performed after the stopper 110 is detached. Then, the vessel 120 and the tube 200 are connected to each other while preventing the contents from leaking, and the inside of the vessel 120 and the inside of the tube 200 communicate with each other so that the contents can flow therethrough.

### 3.4. Step of Moving Magnetic Particles

By undergoing the above-described steps, the magnetic particles M having the nucleic acid adsorbed thereon in the vessel 120 are brought to a state where they can flow through the tube 200. As the method of introducing the magnetic particles M having the nucleic acid adsorbed thereon into the tube 200, a method utilizing a gravitational force or a centrifugal force may be used, and there is no particular limitation on the method, however, in this embodiment, a method in which a magnetic force is applied from the outside of the vessel 120 and the tube 200 is used. A magnetic force can be applied using, for example, a permanent magnet, an electromagnet, or the like, however, from the viewpoint that heat is not generated and so on, it is more preferred to apply a magnetic force using a permanent magnet. In the case of using a permanent magnet, the magnet may be moved by an operator with hand or by using a mechanical device or the like. The magnetic particles M have a property to be attracted by a magnetic force, and therefore, by utilizing the property, the magnetic particles M are moved from the inside of the vessel 120 to the tube 200 by changing the relative position of the permanent magnet to the vessel 120 and the tube 200. By doing this, the magnetic particles M are moved from the first plug 10 to the fourth plug 40 by sequentially passing the particles through the respective plugs. The retention time of the magnetic particles M in each plug when the magnetic particles M pass through the plug is not particularly limited, and also the magnetic particles M may be moved reciprocatively along the longitudinal direction of the tube 200 within the same plug.

### 3.5. Step of Eluting Nucleic Acid

When the magnetic particles M reach the fourth plug 40, the nucleic acid adsorbed on the magnetic particles M is eluted in an eluate in the fourth plug 40 by the action of the eluent. Further, by performing an ultrasound treatment of the magnetic particles M having the nucleic acid adsorbed thereon, the elution amount of the nucleic acid can be increased. The treatment time with ultrasound is not particularly limited, but can be generally set to about 1 to 5 minutes. By undergoing this step, the nucleic acid is eluted with the eluent from the sample, and the nucleic acid is brought to a state of being extracted from the sample. Incidentally, as shown in this embodiment, in the elution step, the elution amount is increased in the case of performing the ultrasound treatment than in the case of performing heating.

### 3.6. Operation and Effect

According to the nucleic acid extraction method of this embodiment, a nucleic acid can be easily extracted in an extremely short time. In the nucleic acid extraction method of this embodiment, by moving the magnetic particles M having a nucleic acid adsorbed thereon in the tube 200 and performing the ultrasound treatment of the magnetic particles M in the eluate, the eluate containing the nucleic acid at a high concentration can be obtained. According to the nucleic acid extraction method of this embodiment, the time and labor required for a pretreatment for PCR can be largely reduced.

3.7. Step of Ejecting Fourth Plug from Tube

The nucleic acid extraction method of this embodiment may include a step of ejecting the fifth plug 50 and the fourth plug 40 from the end of the tube 200 on the other side of the end to which the vessel 120 is connected by deforming the vessel 120.

This step can be performed by deforming the vessel 120 after the “3.5. Step of Eluting Nucleic Acid”. When the fourth plug 40 is ejected, the fifth plug 50 is ejected first. The stopper 110 which seals the tube 200 on the fifth plug 50 side is detached prior to this step to open the end of the tube 200 on the fifth plug 50 side.

When the vessel 120 is deformed by applying an external force thereto to increase the internal pressure, the respective plugs are moved from the first plug 10 side to the fifth plug 50 side in the tube 200 due to the pressure. By doing this, the fifth plug 50 and the fourth plug 40 are ejected in this order from the end of the tube 200 on the fifth plug 50 side. The third plug 30 may be ejected, however, the second plug 20 is prevented from being ejected. In this case, for example, when the volume of the third plug 30 is set to be larger than those of the other plugs so that the length of the third plug 30 in the longitudinal direction of the tube 200 is made longer, the second plug 20 is easily prevented from being ejected.

The fourth plug 40 and the fifth plug 50 are ejected into a reaction vessel for PCR. Therefore, in the reaction vessel for PCR, the eluate and the oil are dispensed. However, the oil generally does not affect the PCR reaction, and therefore, it is also possible to place the same type of oil as the oil of the fifth plug 50 in the reaction vessel for PCR in advance. In this case, when this step is performed in a state where the end of the tube 200 is in the oil, the eluate containing the target nucleic acid can be introduced into the reaction vessel for PCR without being in contact with the outside air. In the case where the nucleic acid extraction method of this embodiment includes this step, the eluate containing the target nucleic acid can be easily dispensed into, for example, a reaction vessel or the like for PCR.

In the case where a cDNA is amplified by using this eluate, if a reaction system in a reverse transcription step and/or a nucleic acid amplification step contains a large amount of the eluate containing the eluted nucleic acid, it becomes also easy to amplify the nucleic acid contained therein in a very small amount. Accordingly, such a reaction system contains the eluate in an amount of preferably 50% or more, more preferably 70% or more, further more preferably 90% or more, and most preferably 100%, that is, it is most preferred to add a reagent for the reaction in advance to the eluent for eluting the nucleic acid.

In general, as the ratio of the eluate in the reaction system is increased, carry-over of ethanol contained in the solution used immediately before the washing step, for example, the adsorbing liquid and/or the second washing liquid inhibits the subsequent reverse transcription reaction or nucleic acid amplification reaction, and therefore, the final yield of the cDNA is decreased, however, in the nucleic acid amplification method according to the invention, by using an acidic solution as the first washing liquid, a decrease in the yield can be prevented.

### 3.8. Modifications

### 3.8.1. Modification of Step of Moving Magnetic Particles

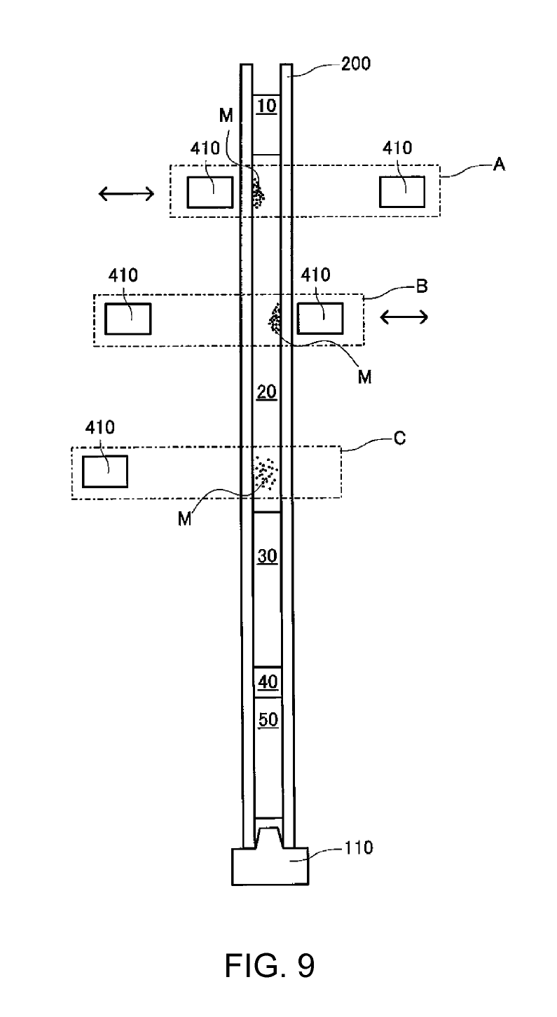

FIG. 9 is a schematic view for illustrating a modification of the nucleic acid extraction method of this embodiment.

In the above-described “3.4. Step of Moving Magnetic Particles”, it is described that by applying a magnetic force to the magnetic particles M from the outside, the magnetic particles M are moved from the first plug 10 to the fourth plug 40 by passing the magnetic particles M through the respective plugs. However, when the magnetic particles M are moved to the second plug 20, by changing the magnetic force to be applied from the outside, the magnetic particles M may be oscillated in the second plug 20 or may be repeatedly aggregated and dispersed. By doing this, the effect of washing the magnetic particles M with the first washing liquid in the second plug 20 can be enhanced.

Specifically, as shown in A and B in FIG. 9, in the case where a pair of permanent magnets 410 are used as a magnetic force application unit, by the permanent magnets 410, the magnetic particles M are moved from the vessel 120 and passed through the first plug 10. When the magnetic particles M reach the second plug 20, one of the permanent magnets 410 is moved away from the tube 200, and the other permanent magnet 410 is moved closer to the tube 200 on the side facing the tube 200, the magnetic particles M can be oscillated in the direction intersecting the longitudinal direction of the tube 200 in the second plug 20 (alternately repeating the modes A and B in FIG. 9). By doing this, the effect of washing the magnetic particles M with the first washing liquid in the second plug 20 can be enhanced. The washing of the magnetic particles M in this manner may be applied to the multiple second plugs 20 or the sixth plug 60 when the second plug 20 is divided or the sixth plug 60 is disposed in the tube 200.

Further, as shown in C in FIG. 9, by simply moving the permanent magnet 410 away from the tube 200, the magnetic particles M can be dispersed in the second plug 20. Since the surfaces of the magnetic particles M are hydrophilic, for example, in the second plug 20, even if the magnetic particles M are dispersed by decreasing the magnetic force applied thereto, the magnetic particles M hardly penetrate into the oil of the first plug 10 or the third plug 30, and therefore, this mode may be adopted.

Specifically, the magnetic particles M are moved from the vessel 120 by the permanent magnet 410 to pass the particles through the first plug 10, and when the magnetic particles M reach the second plug 20, the permanent magnet 410 is moved away from the tube 200 to disperse the magnetic particles Min the second plug 20. Then, the magnetic particles Mare moved again by the magnetic force of the permanent magnet 410 to pass the particles through the third plug 30, whereby the magnetic particles M can be guided to the fourth plug 40.

The mode in which the magnetic particles M are oscillated or repeatedly aggregated and dispersed by changing the magnetic force to be applied from the outside in this manner may also be applied in a state where the magnetic particles M are present in the adsorbing liquid in the vessel 120 or in a state where the magnetic particles M are present in the fourth plug 40 (eluent).

3.8.2. Modification of Step of Ejecting Fourth Plug from Tube

In the case where the above-described “3.7. Step of Ejecting Fourth Plug from Tube” is adopted, the magnetic particles M from which the nucleic acid adsorbed thereon has been eluted in the eluate may be present in the fourth plug 40 in this step, however, after the magnetic particles M are moved to any of the first plug 10, the second plug 20, and the third plug 30, or moved into the vessel 120 by applying a magnetic force, this step may be performed. By doing this, the fourth plug 40 can be ejected from the tube 200 in a state where the magnetic particles Mare not contained in the eluate. Further, by determining the plug to which the magnetic particles M are moved to be the second plug 20 or the vessel 120, even when the magnetic force is removed, the magnetic particles M hardly penetrate into the oil of the third plug 30, and therefore, the fourth plug 40 can be more easily ejected from the tube 200.

### 4. EXAMPLES

The relationship between the ultrasound treatment and the elution amount of a nucleic acid will be described.

### 4.1. Example

In the inside of the tube 200 of the nucleic acid extraction kit 2000 shown in FIGS. 7A and 7B, a nucleic acid extraction device including a first plug 10 to a seventh plug 70 was used. For the first, third, fifth, and seventh plugs, dimethyl silicone oil (KF-96L-2cs, manufactured by Shin-Etsu Silicone Co., Ltd.) was used. For the second plug (first washing liquid), a product of Washing Liquid I (5.9 M guanidine thiocyanate, 1.9% Triton X-100, 54 mM Tris-HCl, pH 7.2) was used, for the sixth plug (second washing liquid), Washing Liquid II (5.3 mM Tris-HCl, pH 7.5) was used, and for the fourth plug (eluent), pure water was used. As a nucleic acid extraction reagent, MagExtractor-Viral RNA- (manufactured by Toyobo Co., Ltd.) was used. The volumes of the second plug, the sixth plug, and the fourth plug were set to 25 μL, 25 μL, 1 μL, respectively.

First, in a 3-mL volume polyethylene vessel, 375 μL of a solution or an adsorbing liquid, and 1 μL of magnetic beads were placed. Then, 50 μL of a fluid obtained by wiping the nasal cavity of a patient with influenza A was added to the vessel with a pipette. The vessel was capped and shaken for 30 seconds to stir the contents. Thereafter, the stopper of the tube on the first plug side and the cap of the vessel were detached, and the tube on the first plug side and the opening of the vessel were connected to each other.

A permanent magnet was moved closer to the vessel to gather the magnetic beads on a side wall of the vessel. Subsequently, the permanent magnet was moved to the position of the fourth plug from the first plug in the tube. Then, the permanent magnet was removed. By doing this, the magnetic beads were moved from the vessel to the fourth plug in the tube. While the magnetic beads were moved in the tube, the time when the magnetic beads were retained in the first, third, and seventh plugs in the tube was 3 seconds each, and the time when the magnetic beads were retained in the second plug was 20 seconds, and the time when the magnetic beads were retained in the sixth plug was 20 seconds.

Subsequently, the stopper of the tube on the fifth plug side was detached, and then, the vessel was deformed by hand, whereby the fifth plug and the fourth plug were ejected into a reaction vessel for PCR. Then, by using an ultrasound generating device US-3KS (manufactured by SND Co., Ltd., oscillation: 38 kHz, BLT self-oscillation), the magnetic beads in the reaction vessel for PCR were subjected to an ultrasound treatment at room temperature for 2 minutes at 120 W. Thereafter, by using a magnet, the magnetic beads were gathered on a side wall of the vessel, and the supernatant was recovered by tilting the vessel.

### 4.2. Comparative Example

Nucleic acid extraction was performed in the same manner as Example except that a heating treatment was performed in place of the ultrasound treatment. The temperature in the heating treatment during elution was set in the range from 25° C. to 95° C. in increments of 10° C.

### 4.3. Detection of RNA

Real-time RT-PCR was performed according to the CDC protocol of realtime RTPCR for swine influenza A (H1N1). More specifically, 9 μL, of a solution containing 0.8 μM of a forward primer, 0.8 μM of a reverse primer, 0.2 μM of a probe, 1× SuperScript III, RT/Platinum Taq Mix, and 1×PCR Master Mix was prepared, and thereto, 1 μL of the liquid recovered in Example or Comparative Example was added, followed by stirring. Then, RT-PCR was performed according to the protocol shown in Table 1. The sequences of the primers and the probe are shown below.

Primers:
Primer F:
GAC CAA TCC TGT CAC CTC TGA C
Primer R:
AGG GCA TTT TGG ACA AAG CGT CTA
Probe:
TaqMan probe:
FAM-TGC AGT CCT CGC TCA CTG GGC ACG-TAMRA

TABLE 1

| | |
|---|---|
| Reverse transcription | 50° C., 30 minutes |
| Activation of Taq | 95° C., 2 minutes |
| PCR (50 cycles) | 95° C., 15 seconds |
| | 55° C., 30 seconds |

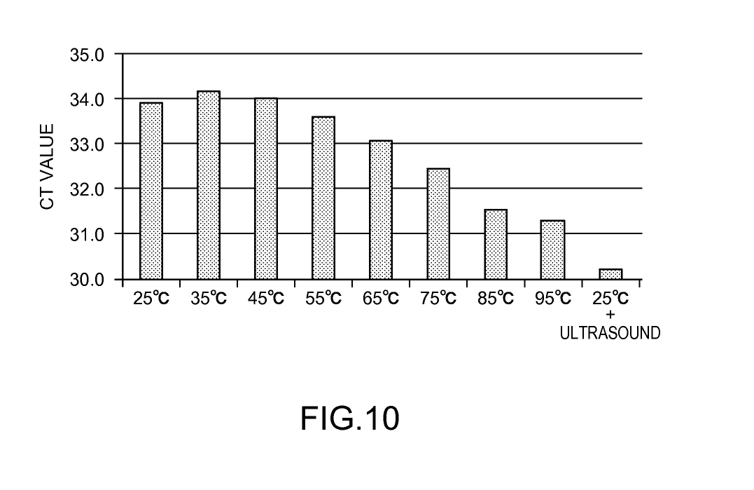

The results expressed as Ct values are shown in FIG. 10. As shown in FIG. 10, the Ct value is smaller in the case of performing the ultrasound treatment at room temperature by about one to four cycles than in the case of performing the heating treatment. In this manner, the elution efficiency is higher in the case of performing an ultrasound treatment in the elution step than in the case of performing a heating treatment.

The invention is not limited to the above-described embodiments, and further, various modifications can be made. For example, the invention includes substantially the same configurations (for example, configurations having the same functions, methods, and results, or configurations having the same objects and effects) as the configurations described in the embodiments. In addition, the invention includes configurations in which parts which are not essential in the configurations described in the embodiments are substituted. In addition, the invention includes configurations that exhibit the same operations and effects as those of the configurations described in the embodiments, or configurations that can achieve the same objects. In addition, the invention includes configurations in which well-known techniques are added to the configurations described in the embodiments.

The entire disclosure of Japanese Patent Application No. 2014-036415, filed Feb. 27, 2014 is expressly incorporated by reference herein.

What is claimed is:

1. A nucleic acid amplification method, comprising:
adsorbing a nucleic acid on fine particles by mixing an adsorbing liquid and the fine particles with a sample containing the nucleic acid;
washing the fine particles having the nucleic acid adsorbed thereon with a first washing liquid;
eluting the nucleic acid adsorbed on the fine particles in an eluate by applying ultrasound to the fine particles having the nucleic acid adsorbed thereon; and
performing a nucleic acid amplification reaction of the nucleic acid in the eluate.

2. A nucleic acid amplification method, comprising:
adsorbing a nucleic acid on fine particles by mixing an adsorbing liquid and the fine particles with a sample containing the nucleic acid;
washing the fine particles having the nucleic acid adsorbed thereon with a second washing liquid;
washing the fine particles having the nucleic acid adsorbed thereon after being washed with the second washing liquid with a first washing liquid;
eluting the nucleic acid adsorbed on the fine particles in an eluate by applying ultrasound to the fine particles having the nucleic acid adsorbed thereon; and
amplifying the nucleic acid.

3. The nucleic acid amplification method according to claim 1, wherein
the nucleic acid is a ribonucleic acid (RNA),
the method further comprises reverse-transcribing the ribonucleic acid eluted in the eluate, thereby synthesizing a cDNA, and
the nucleic acid to be amplified in the nucleic acid amplification is the synthesized cDNA.

4. The nucleic acid amplification method according to claim 2, wherein
the nucleic acid is a ribonucleic acid (RNA),
the method further comprises reverse-transcribing the ribonucleic acid eluted in the eluate, thereby synthesizing a cDNA, and
the nucleic acid to be amplified in the nucleic acid amplification is the synthesized cDNA.

5. A nucleic acid extraction device, comprising:
a tube having a longitudinal direction and internally provided, in the following order, with
a first plug composed of an oil,
a second plug composed of a first washing liquid, which is phase-separated from an oil, and with which fine particles having a nucleic acid bound thereto are washed,
a third plug composed of an oil,
a fourth plug composed of an eluent, which is phase-separated from an oil, and with which the nucleic acid is eluted from the fine particles having the nucleic acid bound thereto, and
a fifth plug composed of an oil;
an ultrasound generating section which applies ultrasound to the fine particles having the nucleic acid bound thereto; and
a vessel which can be connected to and communicate with the tube on the side where the first plug is disposed, and contains an adsorbing liquid for adsorbing the nucleic acid on the fine particles.

6. A nucleic acid extraction device, comprising:
a tube having a longitudinal direction and internally provided, in the following order, with
a first plug composed of an oil,
a second plug composed of a first washing liquid, which is phase-separated from an oil, and with which fine particles having a nucleic acid bound thereto are washed,
a third plug composed of an oil,
a fourth plug composed of an eluent, which is phase-separated from an oil, and with which the nucleic acid is eluted from the fine particles having the nucleic acid bound thereto, and
a fifth plug composed of an oil;
an ultrasound generating section which performs an ultrasound treatment of the fine particles having the nucleic acid bound thereto; and
a liquid reservoir section which communicates with the tube on the side where the first plug is disposed, and contains an adsorbing liquid for adsorbing the nucleic acid on the fine particles, wherein
the adsorbing liquid contains an alcohol, and the first washing liquid is acidic.

7. A nucleic acid extraction device, comprising:
a tube having a longitudinal direction and internally provided, in the following order, with
a first plug composed of an oil,
a sixth plug composed of a second washing liquid, which is phase-separated from an oil, and with which fine particles having a nucleic acid bound thereto are washed,
a seventh plug composed of an oil,
a second plug composed of a first washing liquid, which is phase-separated from an oil, and with which the fine particles having the nucleic acid bound thereto and washed with the second washing liquid are washed,
a third plug composed of an oil,
a fourth plug composed of an eluent, which is phase-separated from an oil, and with which the nucleic acid is eluted from the fine particles having the nucleic acid bound thereto, and
a fifth plug composed of an oil; and
an ultrasound generating section which applies ultrasound to the fine particles having the nucleic acid bound thereto.

8. The nucleic acid extraction device according to claim 7, wherein the device further comprises a vessel which can be connected to the tube on the side where the first plug is disposed, and contains an adsorbing liquid for adsorbing the nucleic acid on the fine particles.

9. The nucleic acid extraction device according to claim 7, wherein the device further comprises a liquid reservoir section which communicates with the tube on the side where the first plug is disposed, and contains an adsorbing liquid for adsorbing the nucleic acid on the fine particles.

10. A nucleic acid amplification reaction cartridge, comprising:
the nucleic acid extraction device according to claim 5; and
a nucleic acid amplification reaction vessel, which communicates with the tube on the side where the fifth plug is disposed, and contains an oil.

11. A nucleic acid amplification reaction cartridge, comprising:
the nucleic acid extraction device according to claim 6; and
a nucleic acid amplification reaction vessel, which communicates with the tube on the side where the fifth plug is disposed, and contains an oil.

12. A nucleic acid amplification reaction cartridge, comprising:
the nucleic acid extraction device according to claim 7; and
a nucleic acid amplification reaction vessel, which communicates with the tube on the side where the fifth plug is disposed, and contains an oil.

13. The nucleic acid amplification reaction cartridge according to claim 10, further comprising a tank, which communicates with the tube on the side where the first plug is disposed, and from which the fine particles are introduced into the tube.

14. A nucleic acid amplification reaction kit, comprising: the nucleic acid extraction device according to claim 7; and a tank from which the fine particles are introduced into the tube.

15. A nucleic acid extraction device, comprising:
a tube having a longitudinal direction and internally provided, in the following order, with
a first plug composed of an oil,
a sixth plug composed of a second washing liquid, which is phase-separated from an oil, and with which fine particles having a nucleic acid bound thereto are washed,
a seventh plug composed of an oil,
a second plug composed of a first washing liquid, which is phase-separated from an oil, and with which the fine particles having the nucleic acid bound thereto and washed with the second washing liquid are washed,
a third plug composed of an oil,
a fourth plug composed of an eluent, which is phase-separated from an oil, and with which the nucleic acid is eluted from the fine particles having the nucleic acid bound thereto, and
a fifth plug composed of an oil; and
an ultrasound generating section which applies ultrasound to the fourth plug.


SEQUENCE LISTING (3 sequences)

SEQ ID NO: 1 - DNA, 22 bp, Influenza A virus
gaccaatcctgtcacctctgac

SEQ ID NO: 2 - DNA, 24 bp, Influenza A virus
agggcattttggacaaagcgtcta

SEQ ID NO: 3 - DNA, 24 bp, Influenza A virus
tgcagtcctcgctcactgggcacg